(19) United States
(12) Patent Application Publication
(10) Pub. No.: US 2002/0133111 A1
(43) Pub. Date: Sep. 19, 2002

(54) Neuro-thrombectomy catheter and method of use

(76) Inventor: John H. Shadduck, Tiburon, CA (US)

Correspondence Address: John H. Shadduck, 1490 Vistazo West, Tiburon, CA, 94920, US

(21) Appl. No.: 10/101,147

(22) Filed: Mar. 16, 2002

Related U.S. Application Data
(60) Provisional application No. 60/277,068, filed on Mar. 19, 2001.

(51) Int. Cl. A61N 1/30

(52) U.S. Cl. 604/19

(57) ABSTRACT

A microcatheter for removing thromboemboli from cerebral arteries in patients suffering from ischemic stroke. The microcatheter provides an extraction lumen that can be scaled to a very small diameter that is still capable of extracting and emulsifying thrombus without clogging the channel. The microcatheter of the invention uses a series of spaced apart energy application mechanisms along the entire length of the catheter's extraction lumen to develop sequential pressure differentials to cause fluid flows by means of cavitation, and to contemporaneously ablate embolic materials drawn through the extraction lumen by cavitation to thereby preventing clogging of the lumen. The catheter system thus provides a functional high-pressure extraction lumen that is far smaller than prior art catheter systems. Preferred mechanisms for energy delivery are (i) a laser source and controller coupled to optic fibers in the catheter wall or (ii) an Rf source coupled to paired electrodes within the extraction lumen. Each energy emitter can apply energy to fluid media in the extraction channel of the catheter—wherein the intense energy pulses can be sequentially timed to cause fluid media flows in the proximal direction in the channel.

### CROSS-REFERENCE TO RELATED APPLICATIONS

This application claims priority from Provisional U.S. Patent Application Ser. No. 60/277,068 filed Mar. 19, 2001 (Docket No. S-AZUR-002) having the same title as this disclosure, which is incorporated herein by reference.

### BACKGROUND OF THE INVENTION

1. Field of the Invention

The present invention relates to medical devices and techniques, and more particularly to a type of catheter that can be scaled to very small diameters suitable for the removal of occlusive thromboemboli in ischemic stroke patients. More in particular, the microcatheter of the invention provides a series of energy delivery structures along the entire length of the catheter's extraction lumen (i) to develop sequential high pressure differentials to cause suction at the distal catheter terminus and fluid flows within the lumen, and (ii) to contemporaneously ablate thromboemboli drawn through the very small extraction lumen to preventing clogging thus providing a functional high-pressure thromboemboli extraction lumen that can be far smaller than prior art catheter systems.

2. Description of Related Art

Stroke is the third leading cause of death in the United States (150,000/year) and the leading cause of disability. About 25% of sufferers die as a result of the stroke or its complications, and almost 50% have moderate to severe health impairments and long-term disabilities, including late-life dementia. About 700,000 strokes occur annually in the U.S. and account for over $26 billion/year in treatment and rehabilitation costs. The incidence of stroke is on the rise.

The majority of strokes occur when a blood clot blocks the flow of oxygenated blood to a portion of the brain. This type of stroke—caused by a blood clot blocking a vessel—is called an ischemic stroke which accounts for 83% of all strokes (the remaining 17% being hemorrhagic strokes). Such an ischemic event can occur as either (i) a thrombotic stroke or (ii) an embolic stroke, and the term occlusive thromboemboli is used at times in this disclosure to describe the occlusive material in either form of ischemic stroke.

A thrombotic stroke or cerebral thrombosis (52% of all ischemic strokes) typically is precipitated by an atherosclerotic disease wherein fatty deposits, calcium, and blood clotting factors such as fibrinogen and cholesterol build-up in a cerebral artery. A smaller percentage of thrombotic strokes result from hypertension, and diseases that cause abnormal arterial blood clot formation (thrombosis) such as atrial fibrillation and heart valve replacement. Two classes of thrombosis can occur in thrombotic stroke-large vessel thrombosis and small vessel disease. Thrombotic stroke occurs most often in the large arteries, magnifying the impact and devastation of the disease. Most large vessel thrombosis is caused by a combination of long-term atherosclerosis followed by rapid blood clot formation in a narrowed vessel. The second type of thrombotic stroke (small vessel disease) occurs when blood flow is blocked to a very small arterial vessel. Little is known about the specific causes of small vessel disease, but it is often linked to hypertension.

Embolic stroke (or cerebral embolism) is also caused by a blood clot. However, unlike cerebral thrombosis, the clot originates somewhere other than the brain. Embolic stroke occurs when a piece of clot (an embolus) breaks loose and is carried by the blood stream to the brain. Traveling through the arteries as they branch into smaller vessels, the clot reaches a point where it can go no further and plugs the vessel, cutting off the blood supply. This sudden blockage is an embolism.

Current treatment modalities for ischemic stroke include mechanical intervention or pharmacologic thrombolytic (drug) therapy to disrupt or dissolve the thrombus. Current mechanical interventions can be relatively invasive and are limited in their accessibility to larger vessels. However, most occlusions occur in smaller, more deeply-seated vessels such as the middle cerebral artery. Thrombolytic therapy may be effective but thrombolytics are not indicated for all stroke victims, are not effective on all thrombus. Further, thrombolytic therapy has associated risks, some of which may have severe consequences-particularly hemorrhage. Successful development of a new treatment modality could provide potentially significant benefits to the outcomes of stroke patients, and ultimately improve mortality rates and decrease morbidity, thereby decreasing the cost of rehabilitation and improving the quality of life for stroke patients.

### SUMMARY OF THE INVENTION

The microcatheter according to the present invention provides a type of mechanical intervention to dissolve and extract thrombus, but can also provide an adjunct localized pharmacological thrombolytic therapy. The microcatheter of the invention has a small cross-section for navigating through small cerebral blood vessels-and provides a functional extraction lumen that is far smaller than such lumens in commercially available catheters. Of particular interest, the extraction lumen of the present invention does not rely on a vacuum source coupled to the catheter handle to create suction forces at the distal open end of the catheter, as is typical in prior art catheters.

More in particular, the microcatheter of the invention is provided with an extraction channel that carries a series of high-intensity pressure-creating emitters along the entire length of the channel for creating brief, intense energy differentials along the entire extraction channel. An energy source, such as a laser and a computer controller, or Rf electrodes, are used to deliver pulsed energy to the emitters (i) to create a sequence of pressure differentials to create peristaltic fluid flows in the extraction channel to suction thromboemboli from the targeted site and entrain emboli within the fluid flows, and (ii) to emulsify and ablate thromboemboli along the entire length of the microchannel to prevent clogging of the channel. By this means of operation, the extraction channel can be very small in cross section, for example from 0.1 mm to 1.5 mm.

In one embodiment, the microcatheter system has from 10 to 20 energy emitters along an extraction channel that increases in dimension in the proximal direction. A laser source and controller coupled to the energy emitters allow for millisecond or microsecond sequential depositions of energy from the emitters to fluid media in the extraction channel. Alternatively, an Rf source coupled to paired electrodes can be used to deliver energy to emitter locations. The sequential energy depositions cause a sequence of transient pressure differentials along the extraction lumen to cause a flow of fluid media through the extraction channel, which can be described as a peristaltic fluid flow mechanism. The energy emitters can cause cavitation in fluid which creates pressure waves along the axis of the lumen. The timing of the energy deposition sequence as well as the increasing diameter of the extraction channel causes fluid flow from the working end toward the handle.

The fluid flow in the extraction channel caused by the sequential energy deliveries causes suction forces at the distal open terminus of the extraction channel that draws thrombus and emboli into the channel. Of particular interest, the plurality of emitters are closely spaced in the distal region of the extraction channel to apply energy to occlusive materials then entrained in the fluid flow within the extraction channel. The emitters then ablate and fragment such thrombus and emboli multiple times until the extraction channel widens, thus eliminating the chance of emboli clogging the very small dimension channel.

In another embodiment, the microcatheter sleeve carries a fluid inflow channel to carry fluid media to the working end to insure adequate levels of fluid flow within the extraction channel and to entrain emboli in the flows. In another embodiment, the system provides a pressure regulator at the handle end of the catheter to reduce proximal fluid flow velocities in the extraction channel by applying backpressure since the flow velocity may become too high. The pressure regulator also can provide negative pressure source at the catheter handle, for example, to induce fluid flows and fill the catheter with fluid media before an energy delivery sequence is commenced to cause peristaltic-type flows.

In another embodiment, the microcatheter uses a fluid inflow channel to deliver any thrombolytic agent (e.g., Reteplase, Streptokinase, Alteplase, and rt-PA, etc.) to the targeted thromboemboli to assist in removal of the occlusion.

In general, the microcatheter of the invention provides a very small diameter working end that provides for high-pressure fluid flows in a small cross-section extraction channel to remove and ablate occlusive thromboemboli from small cerebral arteries in a stroke patient.

The microcatheter provides an extraction channel with a plurality of sequentially actuated energy emitters for creating successive pressure differentials to cause peristaltic fluid flow within the extraction channel.

The catheter system provides means for causing cavitation in fluid media within the extraction channel to apply energy to fluids and entrained occlusive materials to emulsify, fragment and ablate embolic particles.

The catheter system provides pulsatile fluid flows to remove occlusive thromboemboli from a targeted site in a blood vessel.

The catheter system provides a fluid inflow system to deliver thrombolytic agents to thrombus that occludes a blood vessel.

### BRIEF DESCRIPTION OF THE DRAWINGS

Other objects and advantages of the present invention will be understood by reference to the following detailed description of the invention when considered in combination with the accompanying Figures, in which like reference numerals are used to identify like components throughout this disclosure.

FIG. 1 is a plan view of a Type “A” microcatheter of the present invention showing the location of spaced apart energy emitters in the extraction channel together with a block diagram of an exemplary energy source.

FIG. 2 is a perspective cut-away view of the working end of the catheter of FIG. 1 corresponding to the present invention taken along line 2-2 of FIG. 1.

FIG. 3A is a sectional representation of a portion of catheter sleeve similar to FIG. 1 showing an extraction channel that increases in cross-section in the proximal direction.

FIG. 3B is a sectional representation of an alternative catheter sleeve similar to FIG. 3A showing an extraction channel that increases in cross-section in the proximal direction.

FIG. 4 is a plan view of an alternative Type “A” catheter similar to FIG. 1 but showing alternative spaced apart locations of energy emitters in the extraction channel.

FIG. 5 is a graphic illustration of a portion of the catheter's extraction channel of FIG. 2 showing sequential applications of energy to fluid media within the extraction channel illustrating cavitation and differential pressures created thereby to induce fluid flows and suction forces at the distal terminus of the extraction channel.

FIG. 6A is a timeline showing one sequence of energy deliveries to the spaced apart energy emitters in the extraction channel, together with energy levels, in accordance with the method of the invention to cause fluid flows, suction forces and emulsification of thromboemboli.

FIG. 6B is an alternative timeline showing a sequence of energy deliveries and energy levels applied by the energy emitters in accordance with the method of the invention to cause fluid flows, suction forces and emboli emulsification.

FIG. 6C is another timeline of energy deliveries and energy levels applied by the energy emitters in accordance with the method of the invention to cause fluid flows, suction forces and emboli emulsification.

FIG. 6D is yet another timeline of energy deliveries and energy levels in accordance with the method of the invention to cause fluid flows, suction forces and emboli emulsification.

FIGS. 7A-7B are graphic representations of the steps of practicing the principles of the invention utilizing the catheter of FIGS. 1-2:

FIG. 7A being a view of a branch in a cerebral artery that is blocked by thrombus; and

FIG. 7B being a view of pulsed energy applications to media in the extraction channel of the catheter (i) to create a sequential pressure differentials in the extraction channel to cause fluid flows and suction forces; and (ii) to apply energy to fluids and entrained thromboemboli to emulsify, fragment and ablate such embolic materials.

FIG. 8 is a plan view of a Type “B” microcatheter showing spaced apart energy electrical-discharge emitters together with a block diagram of an electrical source and vacuum source coupled to the proximal end of the catheter.

FIG. 9A is a perspective cut-away view of a portion of the catheter of FIG. 8 showing first and second electrodes of a single emitter.

FIG. 9B is a cut-away view similar to that of FIG. 9A showing an alternative arrangement of first and second electrodes.

FIG. 10 is a cut-away view of an alternative Type “B” microcatheter using a series of piezoelectric modules used as energy emitters to develop peristaltic fluid flows in a catheter extraction channel.

### DETAILED DESCRIPTION OF PREFERRED SYSTEM EMBODIMENTS

1. Type “A” Neuro-Thrombectomy Catheter System.

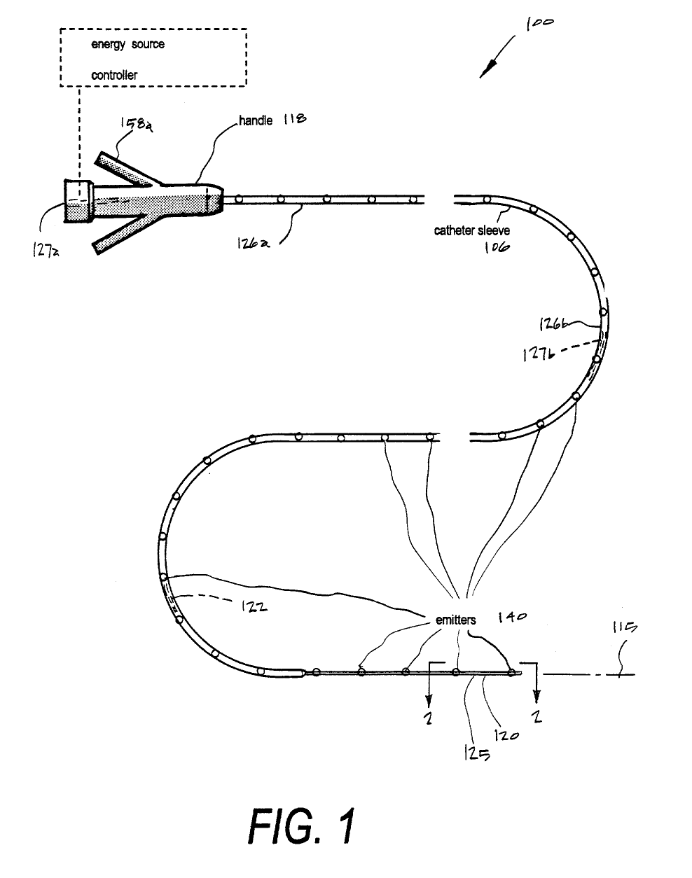

Referring to FIGS. 1 & 2, a Type “A” microcatheter system 100 corresponding to the invention is shown having a thin-wall catheter body or sleeve member 106 that extends along axis 115 from a proximal handle or manifold 118 to distal working end 120 with interior extraction lumen or microchannel 122 extending therethrough. The microcatheter sleeve is fabricated utilizing technology known in the art to provide catheter walls 124 with predetermined flexibility characteristics that can allow precise intravascular navigation, pushability and trackability.

The microcatheter of the invention defines a distal sleeve portion 125 that can have a much smaller cross section than currently available catheters for accessing a targeted neuro-thrombectomy site—while still providing extraction (fluid suction) channel functionality. The exemplary microcatheter of FIG. 1 is adapted for navigating cerebral vasculature with distal sleeve portion 125 (or working end portion) having an outer diameter (OD) ranging from about 0.5 mm to 1.8 mm. Somewhat smaller catheter cross-sections are possible depending on the type of energy emitters and their locations within the extraction lumen 122 (described below), and thus the scope of the invention is particularly adapted to microcatheters having an interior extraction channel with a cross-section ranging between about 0.1 mm and 1.5 mm. More preferably, the interior extraction channel has a cross-section ranging between about 0.2 mm and 1.0 mm.

In the exemplary embodiment of FIG. 1, the distal catheter sleeve portion 125 has a smaller cross-section than the proximal end and medial sleeve portion, 126a and 126b, respectively. Likewise, the medial portion 127b of the extraction channel 122 increases in diameter as further described below to open proximal end 127a. The catheter of the invention also can have a constant OD for introducing through the lumen of a larger catheter already advanced endovascularly. Alternatively, the microcatheter of the invention can itself serve as a guide member (or guidewire) for a larger diameter catheter that provide additional functionality. It should be appreciated that catheters with larger cross sections fall within the scope of the invention.

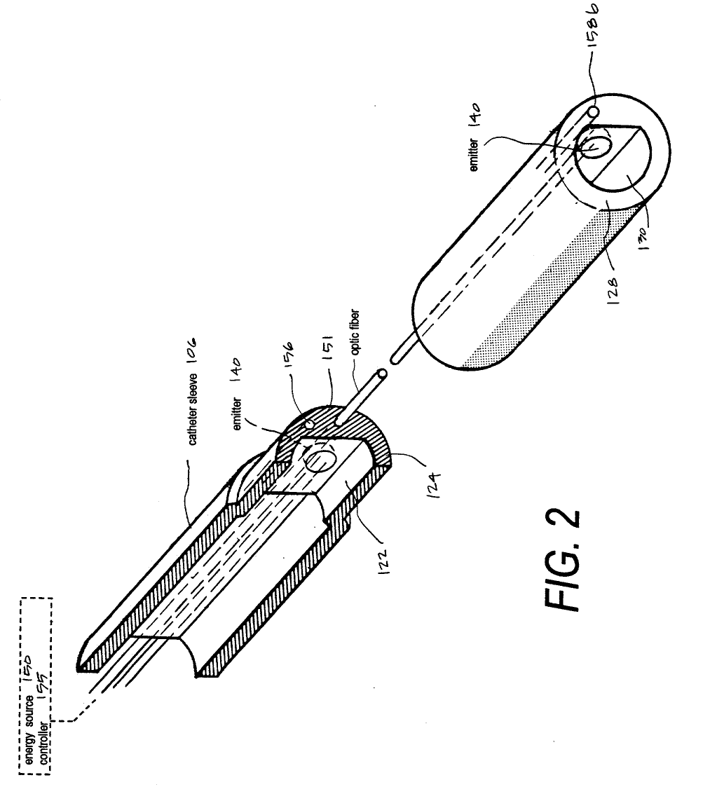

FIG. 2 shows a cut-away view of a portion of the distal end of catheter sleeve 106 defining an engagement surface 128 about the distal open terminus 130 of the extraction channel 122. The engagement surface 128 is adapted to be pushed into substantial contact with targeted thrombus t, or to be navigated into very close proximity to the targeted thrombus, to thereafter utilize the energy application method of the invention to emulsify and suction the occlusive thrombus from the targeted site.

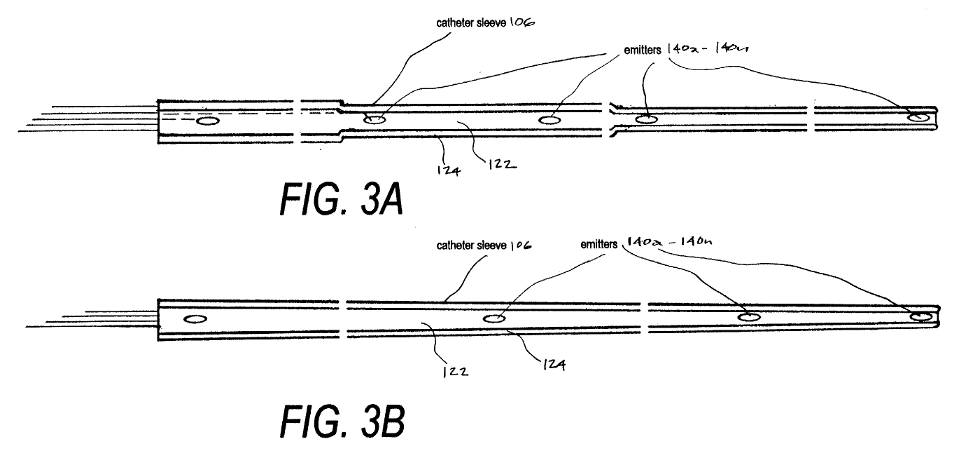

FIGS. 3A-3B each show a schematic view of an exemplary catheter sleeve 106 similar to that of FIGS. 1-2 with an extraction channel 122 extending therethrough for carrying fluid flows. The interior extraction channel has an open (first) proximal end 127a at catheter handle 118 (see FIG. 1) and an open (second) distal terminus 130. The catheter sleeve 106 can be extruded of a flexible material, such as high density polyethylene, polyurethane, PTFE, polyolefin, Hytrel® or another suitable material known in the art of catheter fabrication, with or without a braid reinforcement. The wall 124 of catheter sleeve 106 can be of any suitable thickness and fabrication to insure that channel 122 does not collapse as the sleeve flexes. As described above, the internal passageway 122 of the catheter sleeve has an inner diameter (ID) that can range from about 0.10 mm to 1.5 mm that cooperates with the selected outer diameter—with the interior channel 122 adapted to provide multiple functionality.

Of particular interest, the channel 122 carries energy emitters 140 for creating substantially high-pressure extraction forces or suction forces to extract occlusive thrombus and emboli from the targeted site. Further, the microchannel 122 utilizes the energy emitters 140 to continuously emulsify emboli entrained in fluid flows to prevent clogging of the channel. An exemplary catheter for treating for an ischemic stroke patient can have an overall length of about 150-200 cm. for introduction from the patient's groin. Preferably, a shorter length catheter is used along with a closer percutaneous access to a cerebral artery. Similarly, other shorter lengths of instrument sleeve (whether rigid or flexible) may be provided for treatment of occlusions at other targeted endoluminal sites. In another aspect of the invention, the interior microchannel 122 in larger diameters can comprise a lumen for passing over a guidewire.

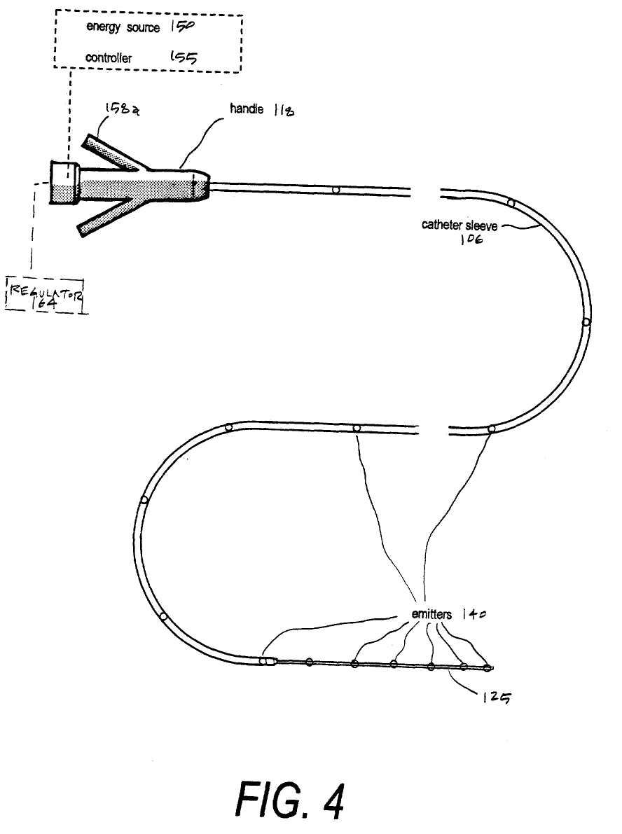

As shown in FIGS. 3A-3B, a Type “A” embodiment has an extraction channel 122 that increases in diameter in the proximal direction to the maximum extent possible for any length of instrument body 106. As shown schematically in FIG. 3A, the extraction channel 122 can increase in diameter step-wise in its medial portion 127 along the length of the lumen, or the channel 122 can increase in diameter in a continuous taper along its length together with the catheter OD (see FIG. 3B). In one embodiment shown in FIG. 1, the extraction channel carries a plurality of energy emitters 140a-140n that are substantially equally spaced apart. In an alternative embodiment depicted in FIG. 4, the extraction channel 122 carries energy emitters 140a-140n with closer spacing in the distal region 125 of the catheter sleeve and wider spacing the proximal direction, for reasons described below.

FIGS. 3A-3B & 5 show cut-away views of a portion of catheter sleeve 106 that depicts a plurality of energy emitters 140a-140n (where n is an integer indicating the number of emitters) carried in catheter walls 124. The energy emitters 140 (collectively) also are described herein as pressure-creating mechanisms since that best describes the functionality of the emitters. Each energy emitter 140 is adapted to apply energy to fluid media m (e.g., blood or introduced fluids) flowing within the extraction lumen 122 for the purpose of accomplishing either of two objectives, or in most cases both objectives. The first or principal purpose of the array of spaced apart energy emitters 140 is to apply sufficient energy in the form of bi-polar stress waves to flowable media m within the extraction channel 122 to cause cavitation—which thereby delivers mechanical energy capable of emulsifying or ablating pieces of thrombus t or other emboli e entrained in fluid flows within channel 122. The second purpose of the energy emitters 140 is to create a sequence of transient pressure differentials along the extraction lumen 122 to cause, or enhance, the flow of fluid media m in the proximal direction through the extraction channel 122. This function then would eliminate, or limit, the need for any independent vacuum source at proximal end 127a of extraction channel 122 to cause fluid flows through the catheter from the open terminus 130 that engages occlusive thromboemboli.

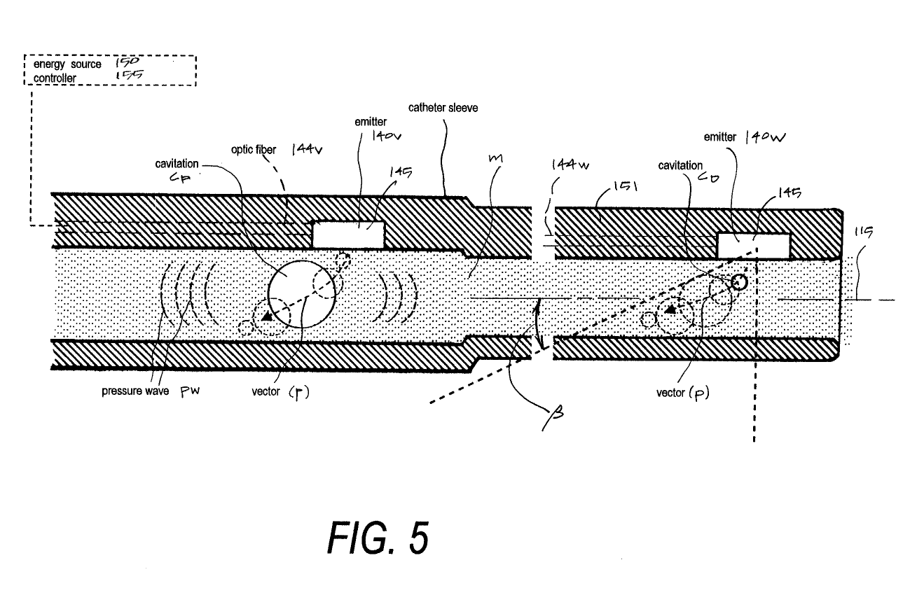

Turning to FIG. 5, two energy emitters 140v and 140w are shown at the distal end of the catheter proximate to open terminus 130. In this embodiment, the light energy emitters comprise the distal end of light channels 144v and 144w together with optional optics (lens, prism, splitter, etc.) collectively indicated at 145 that direct a pulse of light into channel 122. The light channels 144 (collectively) are typically an optic fiber, but can be any form of waveguide known in the art capable of carrying the requisite energy levels, including a fluid core channel that carries a flowable fluid with the required index of refraction to carry a selected wavelength to the particular emitter in question 140. While energy emitters comprising light energy emitters are preferred and described in the practice of the methods of the invention, it should be appreciated that each energy emitter 140 also can be (i) an electrical discharge type of energy emitter, (ii) an ultrasound emitter, or (iii) a microwave emitter—all which can be engineered to be capable of creating cavitation (as described below) that can accomplish the methods of the invention. Piezoelectric elements also fall into the class of energy emitters within the scope of the invention. Some of these alternative types of energy emitters and their cooperating energy sources will be described below. However, to explain the basic operation of one exemplary embodiment of the invention, the system of a plurality of light emitters 140 coupled to a light source 150 is used.

In the catheter of FIGS. 1, 2 & 4, each spaced apart emitter 140a-140n at the distal end of an optic fiber 144a-144n, respectively, carries an optic or mirror 145 known in the art that deflects light propagating down the fiber 144 into extraction channel 122 at an angle β ranging from about 90° to axis 115 to about 10° to the axis (angled to proximal direction, see FIG. 5). FIGS. 3A-3B & 4 show flexible optic fibers carried in the (optional) increased thickness portion 151 of catheter wall 124. Each fiber can have any suitable diameter ranging from about 50 μm to 250 μm., or another larger dimension if requires to meet the energy delivery requirements. The proximal end of each optic fiber 144 is a coupled to a coherent light source 150, which is any suitable laser but also could be a high-intensity pulsed flash lamp that produces a white light (a specified broad wavelength spectrum) as is known in the art. In this embodiment, each emitter 140 is coupled to an independent fiber 144 to allow for sequential firing of the emitters. However, it should be appreciated that a single fiber could connect all emitters 140 to provide concurrent energy delivery to all emitters or to switching system, which is described in a Type “B” embodiment below. The number of emitters may be from 1 to 100 depending on the length of the catheter—and whether the emitters are adapted to deliver energy sequentially or contemporaneously.

The light source 150 is chosen to deliver a selected wavelength (λ) in a short pulse through the optic fiber 144 that is strongly absorbed by media m that is flowing within the extraction channel 122—that is, such media m should have a high absorption coefficient μₐ (cm⁻¹) for the selected λ. Thus, when a pulse of coherent light is delivered very rapidly to the targeted media, the resulting photoabsorption causes thermoelastic expansion of absorbing chromophore molecules or granules in the media causing an intense increase in pressure. For example, blood, thrombus and saline solution are among the targeted media, and the pressure will increase within absorbing media faster than pressure can dissipate from the target (at speed of sound). When there exists a defined or free boundary about a chromophore granule, such as a liquid or gas, the target expands (positive stress) and then can snap-back (negative stress). For example, a laser pulse can that can induce an instantaneous 10° to 50° C. temperature rise in a targeted media theoretically can cause transient pressures of from 10-1000 atmospheres within the target. This process of laser energy absorption in the targeted media can cause formation of a bipolar positive/negative stress wave that propagates into surrounding media. In a liquid or tissue (e.g., blood or thrombus), the bi-polar positive/negative stress wave creates cavitation C within such media causing emulsification, fragmentation or ablation of emboli. In other words, this pulsed energy delivery can emulsify or ablate thromboemboli (pieces of thrombus t, other emboli e) entrained in fluid flow within extraction channel 122. Such emulsification or ablation thereby prevents the extraction microchannel 122—even in very small diameters—from being clogged by embolic material. To accomplish the method of the invention of emulsifying and ablating such materials, the wavelengths from source 150 may range from about 500 mn to 4000 nm, which are suitable for absorption by the potential embolic materials and fluids (e.g., blood, thrombus, emboli, saline or introduced fluids). Lasers that produce wavelengths at suitable powers are well known in the art and need not be described in further detail herein. Laser pulses durations can range from about 1 ns to 1 ms (millisecond), and the fluence is selected to cause cavitation. It should be appreciated that an exogenous chromophore can be added to an introduced fluid media to cooperate with a selected wavelength to provide cavitation at low fluences.

In the Type “A” embodiment as depicted in FIGS. 1-5, the energy emitters 140 also are used to provide the second functionality described previously that relates to the transient creation of a sequence of pressure differentials along the extraction lumen 122 to cause, or enhance, a flow of fluid media m through the extraction channel. More particularly, the schematic sectional view of FIG. 5 shows two emitters 140v and 140w out of a plurality of emitters 140a-140n. The emitters are shown in detail in relation to extraction channel 122 and thick catheter wall portion 151 at its distal region 125. The cooperating light channels (optic fibers) 144v and 144w extend to emitter ports 140v and 140w wherein the light pulse and carried photonic energy therein is directed into media m within extraction channel 122. In this case the emitter carries optic or reflector 145 that directs the light pulse at angle β ranging between about 10° to 90° relative to lumen axis 115 (see FIG. 5). The emitters preferably are more closely spaced in the distal region of the extraction channel 122 to apply energy more closely spaced together to ablate emboli in the narrowest portion of channels 122 (see FIG. 4). By the time the emboli reaches the widened medial portion 127 of extraction channel 122, any emboli would be ablated multiple times and the extraction channel would widen, thus substantially eliminating the chance of clogging that portion of the channel. While the embodiments of FIGS. 2 & 5 show a single emitter at each particular axial location in channel 122, the catheter may provide paired opposing emitters at a particular location to deliver higher energy levels to the media flow, which it is believed could be useful for distal portions of the extraction channel.

FIG. 5 graphically depicts a sequence of energy pulses delivered to media m from spaced apart emitters 140v and 140w showing cavitation within fluid media m. In this case, the energy delivery at the more proximal emitter 140v occurs at time t₁ and the energy delivery at distal emitter 140b occurs at time t_{1+a.u.}, where a.u. is an arbitrary unit time, typically ranging from about 100 microseconds to 100 milliseconds. The cavitation bubble C within the fluid media m expands to a maximum bubble dimension within about 5 to 100 ms, and then collapses and disappears is similar time frame. The graphic representation of cavitation C in FIG. 5 is intended to show the more proximal cavitation indicated at C_{P} has reached its maximum dimension, while the sequentially later distal cavitation C_{D} is just forming and will thereafter expand to its maximum dimension. Each delivery location (proximate to each emitter 140v and 140w) thereby causes a pressure differential in the local fluid media m, which is indicated by pressure waves pw. The expanding cavitation bubble C_{P} causes greater fluid motion in the direction of lesser resistance, which by design is the proximal direction due to the increase in cross-section of extraction channel 122 in the proximal direction. Thus, a single energy pulse causes a photomechanical reaction that will move fluid media m differentially—with a flow impulse being directed generally proximally along axis 115 at each particular emitter location. The sequential applications of energy thus can cause high velocity flows through the length of the channel. In FIG. 5, the distal cavitation is just commencing with phantom views of the cavitation bubble formation and its collapse.

The preferential high-pressure movement of fluids is further enhanced when the light pulse is directed at angle β into the media as indicated in FIG. 5. The cavitation C will itself have and expansion-collapse lifespan as it moves along a directional vector or path indicated at p, thus moving fluid media m in the proximal direction. This photomechanical energy-media interaction thus will accelerate the flow of fluid in the proximal direction. The above form of energy delivery also comprises, in part, a photothermal energy-media interaction since thermal energy plays a role in initial absorption of the photonic energy. The preferred energy parameters described herein are adapted for cavitation or a photomechanical mechanism, but the energy deliveries also could be optimized for photothermal energy-media interaction, for example to assist in the ablation of emboli.

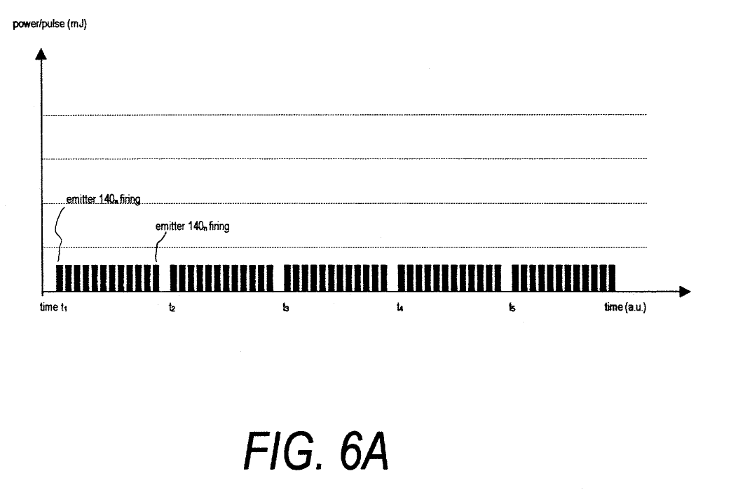

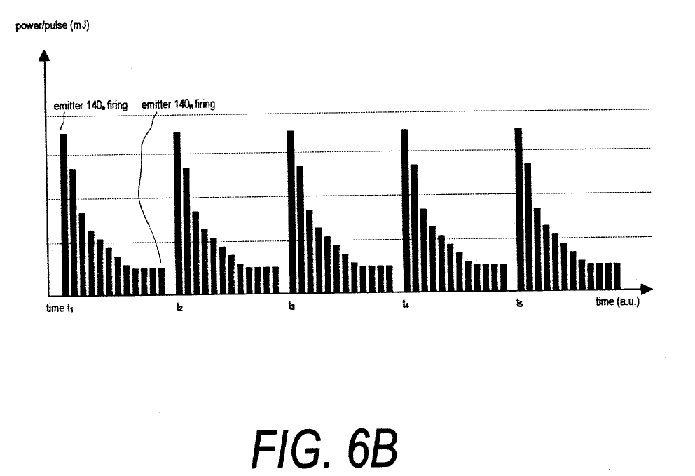

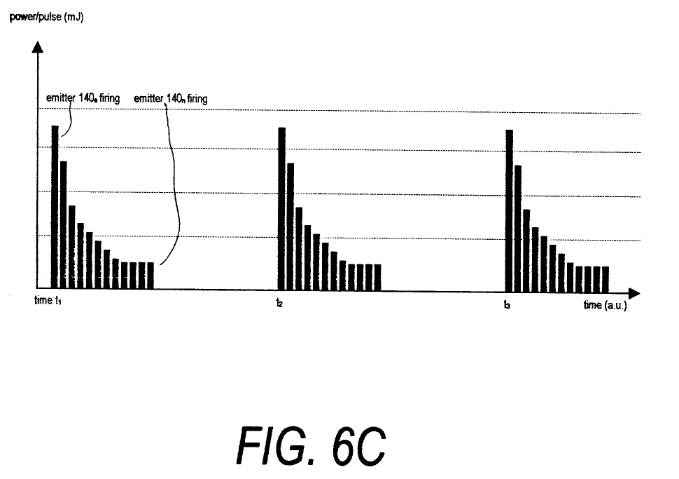

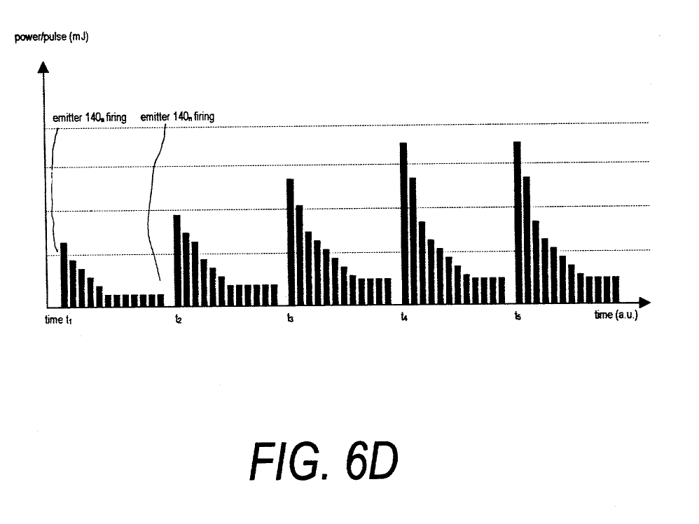

From viewing FIG. 5, it can be seen that the sequential firing of a plurality of energy emitters along the entire length of the extraction channel can accelerate the flow of fluid media m in the proximal direction to develop a high pressure fluid flow having a flow velocity v_{f}. In a typical firing sequence, the proximal energy emitter is fired first to initiate fluid movement in the widest portion of the extraction channel, followed in sequence by each more distal energy emitter, each which moves fluids locally at the site of the emitter. A controller 155 coupled to light source 150 is capable of sequential firing of emitters 140 in a sequence sq that defines a selected time interval between the firing of each individual emitter. The controller 155 can rapidly re-direct a light pulse from source 150 to any particular optic fiber 144 and emitter 140 by any suitable means, for example by using a closed-loop galvanometric optical scanner available from Cambridge Technology, 109 Smith Place, Cambridge, Mass. 02138 within the module that couples the laser source to each fiber optic 144. The controller 155 is further capable of varying the power delivered to each emitter 140, and the profile of such power delivery. Thus, the energy applications occur in a sequence that also defines a pulse duration or interval of energy application, together with an interval between the end of one energy application sequence and the initiation of the next sequence. The controller 155 is capable of a repetition rate of such sequences sq of energy applications at the emitters ranging from about 1 Hz to 500 Hz. Preferably, the repetition rate of sequences sq ranges from about 1 Hz to 100 Hz. It is further believed that by timing each emitter in relation to the phase of the bi-polar wave propagated by an adjacent emitter, that the flow velocity v_{f} in the proximal direction can be enhanced. This factor is dependent on the exact spacing of each emitter 140 relative to an adjacent emitter along the extraction channel. After the controller 155 fires or delivers energy from all emitters 140 in channel 122 in an initial sequence sq, the sequence is repeated leading to a selected repetition rate of firing sequences to cause the continuous flow of media m through extraction channel 122. A typical firing sequence thus is shown in the timeline of FIG. 6A, which applies energy in a manner that will cause, or enhance, a substantially even flow of fluid m through the extraction channel 122. FIG. 6B show a preferred energy delivery sequence wherein the more proximal emitters in the larger diameter channel have higher energy levels than more distal emitters. Another firing sequence is shown in the timeline of FIG. 6C, wherein a time interval is interposed between each firing sequence to cause a slight pulsatile-type suction effect on fluids proximate to terminus 130 of extraction channel 122. Such a micro-pulsatile flow can be useful in emulsifying thrombus engaged by engagement surface 128 of the catheter. Any of these modalities thus can create a rhythmic wavelike movement of pressure differentials through the extraction lumen 122 that comprises a peristaltic mechanism for causing fluid flows within the lumen. A slightly different energy delivery sequence is shown in FIG. 6D wherein energy levels are lower in an initial firing sequence than in a later sequence to slowly build suction and fluid flow velocity v_{f} in extraction channel 122 of the microcatheter.

The microcatheter is particularly adapted for suctioning blood through the extraction channel. The fluid flow may be enhanced by an inflow of a fluid therapeutic agent ta to the working end. For this reason, the microcatheter can have an optional fluid inflow channel 156 in catheter wall 124 (see FIG. 2). The proximal end 158a of channel 156 is coupled to a therapeutic fluid media source 162 (e.g., a bag of saline, etc.) that can provide a very low pressure flow of therapeutic agent ta to a media entrance port 158b at the working end as shown in FIG. 2. The suction forces created by the energy discharges can draw the fluid therapeutic agent ta into the extraction channel to insure there is adequate fluid flow within the channel to entrain emboli and serve as media for cavitation to create the pressure differentials.

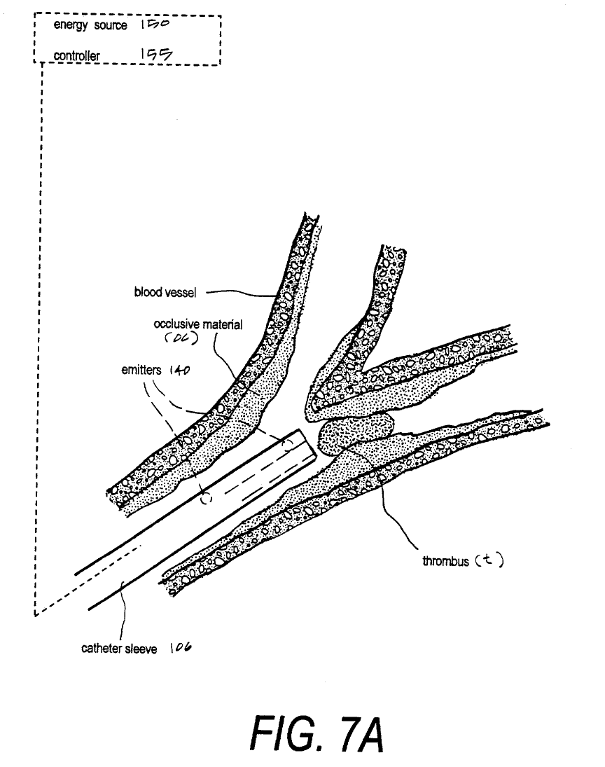

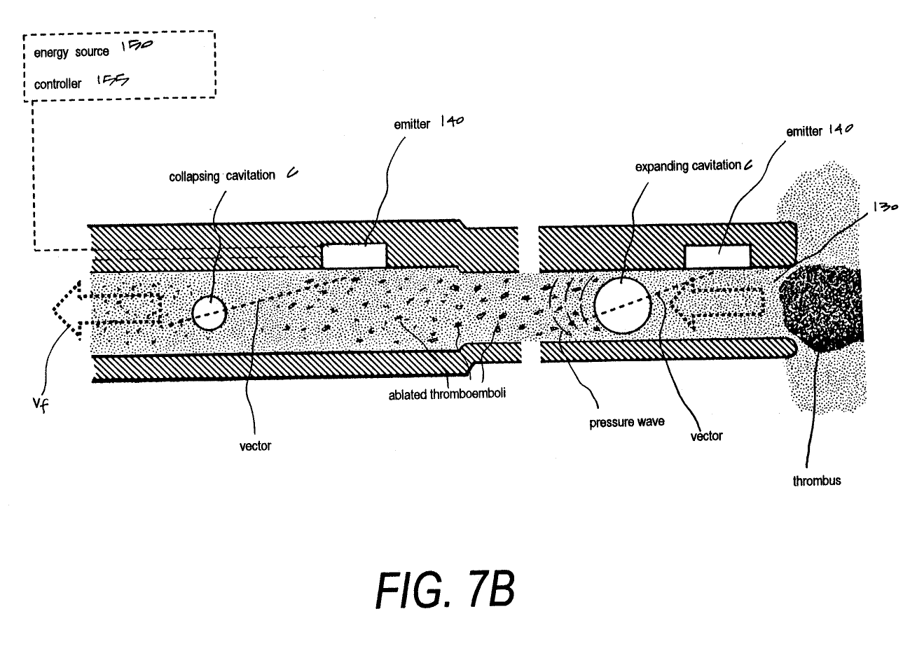

Now turning to FIG. 7A, it can be understood how thromboemboli indicated t that occludes a cerebral artery can be emulsified and extracted through a very small diameter microchannel 122 to practice the method of the invention. FIG. 7A shows a branch in a cerebral artery with occlusive material oc (fatty deposits, calcium, plaque) narrowing the lumen as is common in atherosclerotic disease. The formation of thrombus is indicted at t that blocks blood flow through the branch artery. As depicted in FIG. 7A, the distal end of catheter 100 is navigated to the targeted site under suitable imaging as is known in the art, for example from a femoral access but more preferably from an endovascular access closer to the targeted site. The use of a guidewire is not shown, but may be typical. The physician engages the thrombus t with the engagement surface 128 and open terminus 130 of extraction channel 122. The physician then actuates the controller 155 and source 150 to deliver energy to the emitters 140 in a sequence as described above (see FIGS. 6A-6D) thereby causing suction forces at the distal end of channel 122. FIG. 7B is a graphical illustration of the disruption of the thrombus t and suctioning of thrombus portions into extraction pathway 122 to be entrained in a selected flow velocity v_{f}. FIG. 7B further illustrates that other emboli e can be detached from the occlusive material in the vessel and carried into the extraction pathway. As can be seen in FIG. 7B, the energy delivery from emitter 140 at the distal end of extraction channel 122 causes cavitation C within blood and pieces of thrombus t drawn into the channel thereby emulsifying the thrombus. Also, any emboli e of more solid material can be ablated or fragmented by the energy delivery that causes cavitation thereby preventing such material from clogging the very small cross-section extraction channel.

Another aspect of method of the invention (not shown) includes the optional delivery of a biocompatible fluid therapeutic agent ta (e.g., saline solution) to the working end via at least one media entrance port 158b proximate to the distal engagement surface 128 about terminus 130 of extraction channel 122 (see FIG. 2). This flow of saline can be provided to entrain emboli, dissolve thrombus and provide an additional volume of flowable media to cavitate in an energy delivery sequence to thereby apply energy to extracted materials. In another embodiment, the catheter can provide a fluid inflow port proximate to each emitter to insure adequate fluid media at the site of energy deposition. In another aspect of the invention, the fluid therapeutic agent ta can be any suitable pharmacological agent for causing thrombolysis (e.g., reteplase, etc.) that flows from a media entrance port about the engagement surface 128 of the catheter to dissolve the thrombus.

In another aspect of the invention (not shown), the controller 155 can be operatively connected to a pressure regulator system 164 at the proximal end of extraction channel (see FIG. 4). It is believed that energy applications may cause peristaltic flows at very high pressures such that the proximal fluid flow velocity is higher than desired. Thus, if the pressure differential-induced (peristaltic) movement of media m within extraction channel develops such an overly high velocity v_{f}, the regulator system 164 can decrease the outflow by applying back pressure at the handle 118 of the catheter, or by any other pressure-regulating means known in the art. Thus, the controller 155 can control flow velocity v_{f} by modulating power and sequencing of energy applications, or by modulating outflow volumes and pressures at the handle end of the extraction channel. In a related optional method of the invention (not shown), the controller 155 can add negative pressure (suction) at the proximal end of the extraction channel to initiate or enhance media flows through the extraction channel from an independent source.

2. Type “B” Neuro-Thrombectomy Catheter System.

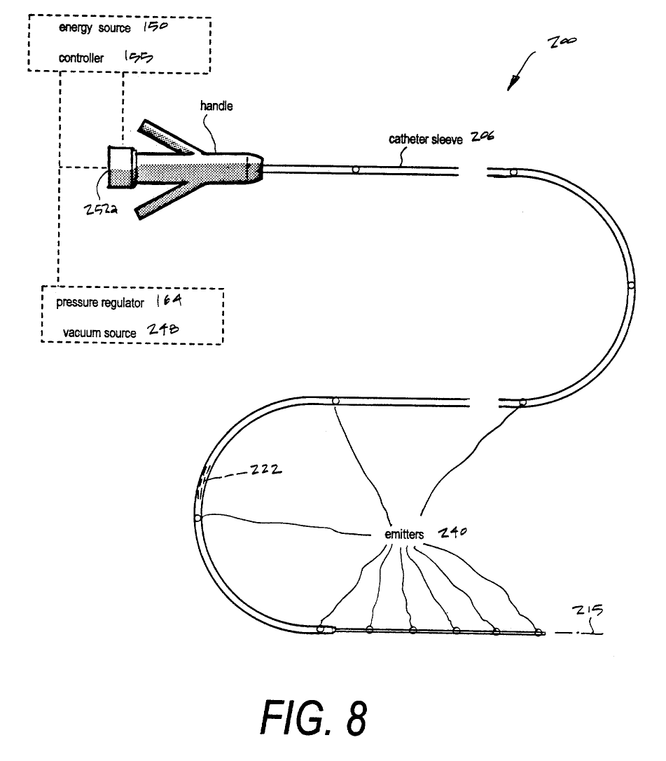

Referring to FIG. 8, the Type “B” microcatheter system 200 corresponding to the invention comprises a catheter body 206 extending along axis 215 that defines interior extraction channel 222 extending the length of the catheter. This embodiment of microcatheter again carries a plurality of energy emitters 240 (collectively) along the length of the extraction channel 222 that serve as cavitation-creating mechanisms. As in the Type “A” embodiment, the energy emitters 240 are adapted to apply energy to fluid media m (e.g., blood, saline) flowing within the extraction lumen 222—but this time for the single purpose of delivering bi-polar stress waves to the media for emulsifying or ablating pieces of thrombus t or other emboli e entrained fluid flows. In this embodiment, the energy emitters 240 are all fired contemporaneously and not relied on to create pressure differentials to cause peristaltic fluid flows. Instead, vacuum source 248 is coupled to the proximal end 252a of the extraction channel 222 to draw the fluid media through the length of the microcatheter. This type of system is best adapted for shorter length extraction channels in a medical device body since the power of the vacuum source, which is limited in a very small diameter lumen, must overcome the pressure waves caused by multiple points of cavitation. Some of such pressure waves will have the tendency to push fluids distally.

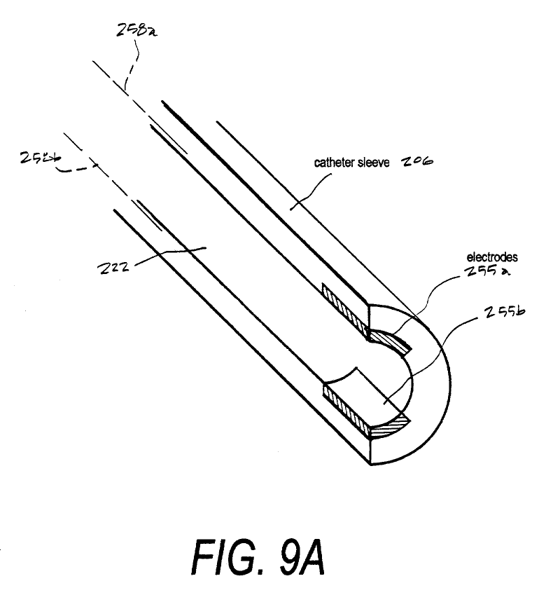

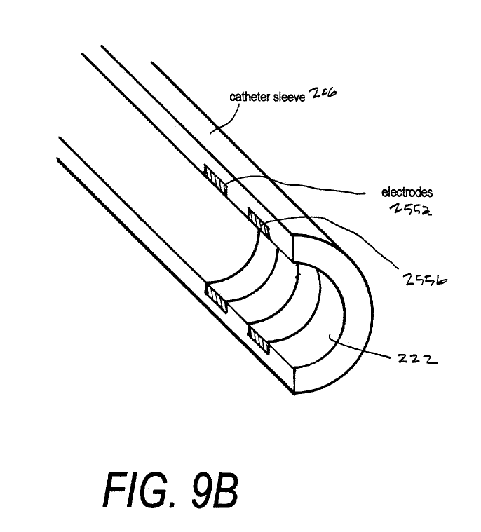

In this Type “B” embodiment, since the plurality of energy emitters 240 are fired contemporaneously, a series of paired electrodes 255a and 255b can function as means for delivering energy to the fluid media by causing an electrical discharge (see FIG. 9A). The plurality of paired electrodes can be coupled to a single pair of leads 256a and 256b that are coupled to an electrical source 260. Each pair of spaced apart electrodes 255a and 255b can be positioned across from one another or axially spaced apart as shown in FIGS. 9A-9B, respectively. It should be appreciated that a single optic fiber could also be used to simultaneously apply energy from each emitter. Either paired electrodes or light-energy emitters can be provided that can apply a differential level of energy at each emitter location with a single level of power input from the remote energy source.

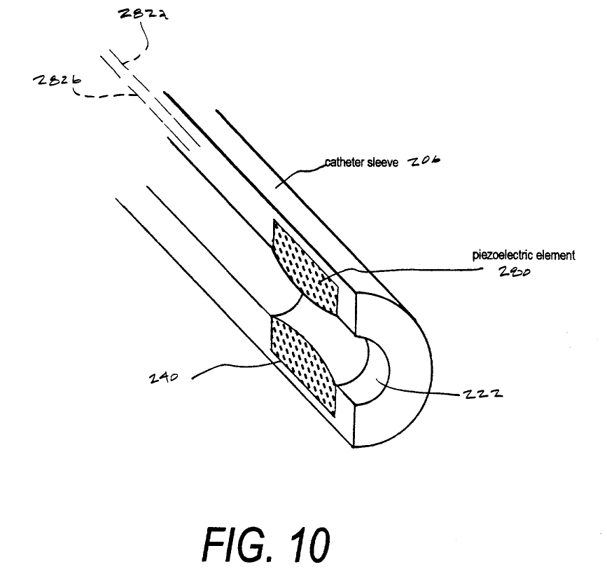

In another Type “B” embodiment shown in FIG. 10, the plurality of energy emitters 240 comprise piezoelectric materials 280 with channel or bore 222 extending therethrough. These piezoelectric materials 280 are coupled to an electrical source via leads 282a and 282b and a controller 285 to cause very rapids oscillations in the diameter of bore 222 thereby delivering energy to fluid media within the bore. Such energy deliveries are easily capable of fragmentation of thrombus and causing peristaltic fluid flows, although investigations are ongoing as to whether the energy levels are capable of causing cavitation.

The catheter my have any suitable radio-opaque markings as are known in the art. In another embodiment (not shown) the distal open terminus 131 of the extraction channel 122 (see FIG. 2) may comprise a single opening or plurality of openings about the end and sides of the distal catheter wherein a further “distal protection” structure is provided, which comprises an occlusion balloon, or mesh that is expanded to an open position by a wire or inflated rim portion, or a perfusion balloon system. Such a catheter working end then would be used for treating narrowed lumens where the “distal protection” structure could be passed beyond the targeted site to prevent any emboli from migrating downstream. The system of the invention then would suction and emulsify thromboemboli, while capturing any fragments that initially migrated distally before being extracted. In such an embodiment, the fluid media inflow ports 158b can be singular or plural and spaced apart proximally from the open channel terminus or termini 131 thereby providing a flow or introduced fluid about the targeted site.

In another embodiment (not shown) the extraction lumen can be fitted with a plurality of one-way flow valves such as flap-type valves or leaf-type valves to prevent fluid flows in the distal direction in the extraction channel 122. Thus, the energy deliveries would direct all forces proximally, as any initial pressure wave pw in the distal direction would close such valves to distal flows.

The system has been described above for use in thrombectomy and other similar endovascular interventions. However, it should be appreciated that a similar system can be used in any body lumen or duct (e.g., ureter, bile ducts, etc.) to cause removal and emulsification of occlusive materials oc. Also, the methods of causing peristaltic flows by sequential spaced apart energy deliveries to fluid media in a microchannel to create pressure differentials (without cavitation) can apply to a microchannel in any medical device, including diagnostic or other chips that have microchannels.

Those skilled in the art will appreciate that the exemplary embodiments and descriptions thereof are merely illustrative of the invention as a whole, and that variations in controlling the duration of intervals of energy delivery, in controlling the repetition rate, and in controlling the amount of energy applied per pulse may be made within the spirit and scope of the invention. Specific features of the invention may be shown in some figures and not in others, and this is for convenience only and any feature may be combined with another in accordance with the invention. While the principles of the invention have been made clear in the exemplary embodiments, it will be obvious to those skilled in the art that modifications of the structure, arrangement, proportions, elements, and materials may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

What is claimed is:

1. A medical catheter, comprising:
a catheter sleeve defining an interior channel extending along an axis between a first end and a second end; and
a plurality of spaced apart pressure-creating emitters exposed to the interior channel; and
an energy source coupled to each pressure-creating emitter for delivering an intense pulse of energy to media within the interior channel.

2. The medical catheter of claim 1 wherein the pressure-creating emitter comprises an optic fiber in said catheter sleeve having with a distal end emitter exposed in said interior channel.

3. The medical catheter of claim 1 wherein the pressure-creating emitter comprises paired electrodes spaced apart in a channel portion of said catheter sleeve.

4. The medical catheter of claim 1 wherein the interior channel has a cross-section ranging from 0.1 mm to 1.5 mm.

5. The medical catheter of claim 1 wherein the interior channel has a cross-section ranging from 0.2 mm to 1.0 mm.

6. The medical catheter of claim 1 further comprising a controller operatively connected to the energy source for controlling parameters of energy deliveries at said emitters, said parameters selected from the class of controlling the timing the energy deliveries and controlling the power of energy deliveries at said emitters.

7. The medical catheter of claim 6 wherein the controller is capable of a repetition rate of energy applications at the emitters ranging from about 1 Hz to 500 Hz.

8. The medical catheter of claim 1 wherein the pressure-creating emitter is selected from the class consisting of light energy emitters, electrical discharge emitters, piezoelectric emitters, ultrasound transducers, and microwave emitters.

9. The medical catheter of claim 1 wherein the pressure-creating emitter are capable of delivering pulses of energy for causing cavitation within fluid media.

10. The medical catheter of claim 1 further comprising a fluid inflow lumen within a wall of the catheter sleeve coupled to remote fluid media source.

11. The catheter of claim 11 further comprising at least one media inflow port in a distal portion of the catheter sleeve that communicates with said inflow lumen.

12. A method for moving fluids in an interior channel of an elongate medical device, comprising the steps of:
(a) providing a device body defining an interior channel extending along an axis between a first end and a second end; and
(b) sequentially actuating a plurality of spaced apart pressure-creating mechanisms along the length of the interior channel thereby sequentially creating transient pressure differentials that move fluids from transiently higher pressure regions to transiently lower pressure regions thereby causing fluid flow within the channel.

13. The method of claim 12 wherein the pressure-creating mechanisms deliver energy causing cavitation in fluids within the interior channel.

14. The method of claim 13 wherein the pressure-creating mechanisms create cavitation that expands and collapses generally along a directional vector within the interior channel.

15. The method of claim 13 wherein said cavitation emulsifies occlusive materials within said fluid flow in the interior channel.

16. The method of claim 15 wherein a controller controls parameters of energy deliveries selected from the class of controlling the timing the sequential actuation of the pressure-creating mechanisms and controlling the power of energy applications among the spaced apart pressure-creating mechanisms.

17. An elongated medical device for endoluminal therapies, comprising:
a member body defining an interior extraction channel extending between a proximal end and an open distal terminus; and
a plurality of spaced energy emitters exposed to said interior extraction channel between said proximal end and said distal terminus, each said emitter comprising paired opposing polarity electrodes; and
an electrical source coupled to said paired electrodes for applying energy to media within the interior channel.

18. The medical device of claim 17 wherein said interior channel has a cross-section of less that 1.0 mm.

19. The medical device of claim 17 further comprising a plurality of one-way flow valves within said interior channel.

20. The medical device of claim 17 further comprising a negative pressure source coupled to a proximal end of said interior channel.
